(19) United States
(12) Plant Patent Application Publication
(10) Pub. No.: US 2002/0184685 P1
(43) Pub. Date: Dec. 5, 2002

(54) Chrysanthemum plant named 'GEDI ONE JAM'

(76) Inventor: Dirk Pieters, Oostnieuwkerke (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,748

(22) Filed: Jun. 4, 2001

(51) Int. Cl. A01H 5/00

(52) U.S. Cl. PLT/291

(57) ABSTRACT

A new and distinct Chrysanthemum plant variety characterized by a large decorative type inflorescence, consistent flowering response to short days, blooming consistently after 49 days of short day length, free branching habit, and pink ray florets which are resistant to fading with age.

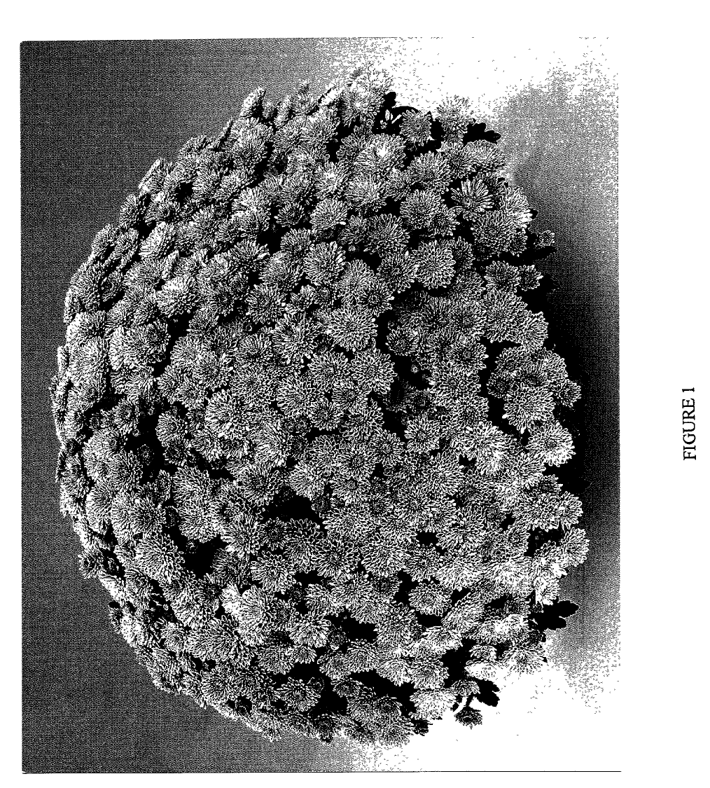

### BACKGROUND OF TEH INVENTION

The present invention relates to a new and distinct cultivar of Chrysanthemum plant, botanically known as Dendranthema morifolium and herein after referred to by the cultivar name ‘GEDI ONE JAM.’

This new cultivar was the result of hybridization of a female parent, ‘Ozenda’ (U.S. Plant Pat. No. 10,419) with a male parent, ‘Maribelle’ (unpatented.) The new cultivar was selected in October 1997.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE JAM’ has not been observed under all possible environmental conditions. The phenotype may vary somewhat with variations in environment such as temperature, daylength, and light intensity, without, however, any variance in genotype.

The following traits have been repeatedly observe and are determined to be the unique characteristics of ‘GEDI ONE JAM.’ These characteristics in combination distinguish ‘GEDI ONE JAM’ as a new and distinct Chrysanthemum:

1. Large decorative type inflorescence;

2. Consistent flowering response to short days, blooming consistently after 49 days of short day length;

3. Free branching habit; and

4. Pink ray florets, resisting fading with age.

Plants of the new Chrysanthemum are similar to plants of the parent cultivars in most horticultural characteristics, however plants of the new cultivar differ from plants of the female parent cultivar ‘Ozenda’ in the following characteristics:

1. The new cultivar naturally blooms 2 weeks earlier;

2. The new cultivar has individual blooms that are more resistant to fading;

3. The new cultivar has many more ray florets than the female parent; and

4. The new cultivar has larger diameter bloom.

The new cultivar also differed from the male parent ‘Maribelle’ in the following characteristics:

1. The new cultivar has a different ray floret color; and

2. The new cultivar naturally blooms 2 weeks earlier.

The new cultivar can be compared to the Chrysanthemum cultivar ‘Jean’ (disclosed in Plant Pat. No. 10,213.) However plants of the new Chrysanthemum differed from the plants of the cultivar ‘Jean’ in the following characteristics:

1. New cultivar naturally blooms approximately 1 week earlier;

2. New cultivar has ray florets that are more pink than lavendar;

3. New cultivar has a more round plant shape; and

4. New cultivar has a slightly larger diameter flower.

### BRIEF DESCRIPTION OF THE PHOTOGRAPHS

The photograph shows a plant of ‘GEDI ONE JAM’ grown in a 2 gallon container. One cutting was used in the pot, planted mid-May and grown under outdoor field conditions.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘GEDI ONE JAM’

Commercial classification: Garden-type Chrysanthemum

Parentage: Hybridization of female parent ‘Ozenda’ (U.S. Plant Pat. No. 10,419) and male parent ‘Maribelle’ (unpatented)

### Propagation

Time to Rooting: 7 to 14 days at approximately 21° C.

Rooting habit: Fine, fibrous

### Plant

Growth Habit: Mounding herbaceous perennial

Height: Approximately 30 cm

Growth rate: Moderate

Branching Characteristics: Free Branching

Length of Lateral Branches: Approximately 25 cm

Number of leaves per lateral branch: Approximately 28

Stem color: Near 137A

### Foliage

Leaf:

Arrangement.—Alternate single

Average Length.—Approximately 8 cm

Average Width.—Approximately 5.2 cm

Shape of blade.—Ovate

Apex.—Acuminate

Base.—Attenuate

Attachment.—Stalked

Margin.—Serrate

Texture of top surface.—Lightly pubescent

Texture of bottom surface.—Lightly pubescent

Color.—young foliage upper side: Near 138A young foliage under side: Near 138A mature foliage upper side: Near 138A mature foliage under side: Near 138A venation upper side: Near 138C venation under side: Near 138C

Petiole:

Average length.—Approximately 3.2 cm

### Bloom

Inflorescence:

Flowering habit.—Induced by darkness period greater than 13.5 hours, approximately 49 days of appropriate day length required to induce and develop blooms

Inflorescence form.—Decorative

Natural flowering season.—Approximately mid-September

Number of in florescences per stem.—Approximately 11

Inflorescence diameter.—Approximately 8.3 cm

Inflorescence depth: 2.3 cm

Inflorescence longevity on plant.—Approximately 24-29 days

Persistence.—Persistent

Ray Florets:

Appearance.—Matte

Texture.—Smooth

Average Number per flower.—Approximately 300

Shape.—Oblanceolate

Margin.—Entire

Apex.—Obtuse

Length.—Approximately 4.2 cm

Width.—Approximately 1.1 cm

Color.—upper surface at first opening: Near 66D upper surface at maturity: Near 62C upper surface at fading: Near 62D under surface at first opening: Near 59D under surface at maturity: Near 62B under surface atfading: Near 62C

Disc florets:

Appearance.—Shiny

Texture.—Smooth

Average number per flower.—Approximately 20

Shape.—Cylindric

Apex.—Obtuse

Average length.—Approximately 1 cm

Average width.—Approximately 0.2 cm

Color.—at first opening: Near 2A at maturity: Near 7A at fading: Near 17D

Peduncle:

Length.—at terminal end (shortest): Approximately 8 cm at lateral end (longest): Approximately 11 cm

Strength.—Moderate

Color.—Near 137C

Habit.—Erect

Inflorescence Bud:

Length.—Approximately 1 cm

Diameter.—Approximately 1 cm

Form.—Globular

Color.—Near 66D

Sepals:

Appearance.—Matte

Texture.—Lightly pubescent

Number.—Approximately 30 to 40

Shape.—Ovate

Margin.—Entire

Apex.—Acute

Color.—upper side: Near 138A under side: Near 138A

### Reproductive Organs

Ray Florets:

Pistillate.—Number of pistils per flower: 1 Stigma shape: 2 branched Stigma color: Near 6A Style color: Near 6D Style length: Approximately 0.6 cm

Disc Florets:

Pistillate.—Number of pistils per flower: 1 Stigma shape: Cylindric Style length: Approximately 0.5 cm

Staminate.—Number of stamens per flower: Approximately 5 Anther shape: Tubular Anther color: Near 6A Pollen color: Near 7A

### Other Characteristics

Disease Resistance: Neither resistance nor susceptibility observed

What is claimed is:

1. A new and distinct variety of Chrysanthemum plant named ‘GEDI ONE JAM’ as herein illustrated and described.
